(19) United States
(12) Patent Application Publication
(10) Pub. No.: US 2006/0062742 A1
(43) Pub. Date: Mar. 23, 2006

(54) Compositions for reduction of human malodor

(75) Inventors: Catherine Cornick Davis, Sharonville, OH (US); Thomas Ward Osborn III, Clifton, OH (US); Kenneth Wallace Miller, Liberty Twp, OH (US); Bruce Elliott Jones, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 11/205,942

(22) Filed: Aug. 17, 2005

Related U.S. Application Data
(60) Provisional application No. 60/612,398, filed on Sep. 21, 2004.

(51) Int. Cl. A61K 35/74; A61K 9/68; C12N 1/20

(52) U.S. Cl. 424/48; 604/360; 424/93.45; 435/252.9

(57) ABSTRACT

A composition and methods for reducing malodor in a human. The composition includes one or more bacteria selected from the group consisting of Lactobacillus iners and all clones with at least 97% sequence similarity to Lactobacillus iners as determined by sequences from 16S rRNA genes. The method includes administering a safe and effective amount of the composition.

### CROSS REFERENCE TO RELATED APPLICATION

This application claims the benefit of U.S. Provisional Application No. 60/612,398, filed Sep. 21, 2004.

### FIELD OF THE INVENTION

This invention relates to bacterial compositions and methods for reducing human malodor.

### BACKGROUND OF THE INVENTION

Human malodor exists in many forms. Halitosis or bad breath is one form of human malodor that can be embarrassing for the individual suffering from such malodor. Previous attempts to solve the problem of halitosis include using a mouthwash and brushing the tongue. Such solutions are less than satisfactory to many individuals suffering from such a problem.

Another type of human malodor is vaginal odor. Numerous types of douches have been used in an attempt to solve the problem of vaginal odor.

Another type of human malodor is urogenital odor. Wipes with fragrances have been used in attempt to cover up the odor with the new more pleasant scent that is being introduced by the wipe.

The above solutions have proven less than satisfactory and thus there is a need to solve the problem of human malodor a different way.

It is believed that the presence of an unhealthy flora on the mucosal surfaces of a human is a cause for malodor. For example, the presence of an unhealthy flora in the mouth leads to bad breath or halitosis. A similar malodor problem exists in other mucosal surfaces of humans such as the vagina, urogenital region and the gastrointestinal tract. By re-establishing a healthy flora malodor associated with an unhealthy flora can be reduced and even eliminated.

Halitosis, or bad breath, is caused by mainly volatile sulfur compounds (VSC) as a result of bacterial breakdown of protein and can be quantitatively and qualitatively measured in the expired oral breath. In eight to ninety percent of cases, halitosis originates from the mucosal and/or dental surfaces mouth due to inadequate plaque control, periodontal disease, dry mouth, faulty restorations, and in particular due to excessive bacterial growth on the posterior third of the dorsal surface of the tongue. In the remaining ten to twenty percent of cases, bad breath is caused by systemic disorders such as hepatic, pancreatic and nephritic insufficiencies, trimethylaminuria, upper and lower respiratory tract infection, medication and cases where gastric content may generate oral malodor.

Utilizing culture-independent molecular tools, microbial profiles of the tongue dorsa from patients with halitosis have been constructed. (Kazor, et al., J Clin Microbiol 41:558-563, 2003). Some of the species most associated with halitosis were Atopobium parvulum and Eucbacterium sulci.

This invention relates to bacterial compositions and methods for reducing human malodor.

### SUMMARY OF THE INVENTION

The present invention relates to a composition and methods for reducing human malodor. The composition comprises one or more bacteria selected from the group consisting of Lactobacillus iners, and all clones also referred to as isolates with at least 97% sequence similarity to Lactobacillus iners as determined by sequences from 16S rRNA genes. The method for reducing human malodor comprises administering one or more bacteria selected from the group consisting of Lactobacillus iners and clones with at least 97% sequence similarity to Lactobacillus iners as determined by sequences from 16S rRNA genes.

### DETAILED DESCRIPTION OF THE INVENTION

As used herein “applicator” refers to a device or implement that facilitates the insertion of a tampon, medicament, treatment device, visualization aid, or other into an external orifice of a human, such as the mouth, vagina, rectum, ear canal, nasal canal, or throat. Non-limiting specific examples of such include any known hygienically designed applicator that is capable of receiving a tampon may be used for insertion of a tampon, including the so-called telescoping, tube and plunger, and the compact applicators, an applicator for providing medicament to an area for prophylaxis or treatment of disease, a spectroscope containing a microcamera in the tip connected via fiber optics, a speculum of any design, a tongue depressor, a tube for examining the ear canal, a narrow hollow pipe for guiding surgical instruments, and the like. Applicator devices such as a toothbrush, cotton and/or Dacron applicator, or a tongue depressor may also be used.

As used herein, the term “deactivation” means to make less toxic or nontoxic.

As used herein, the term “density” is used with its common technical meaning with units of g/cm³ or g/cc. The density may refer specifically to that of a specific region or feature of the tampon as noted. The density will be measured, unless otherwise noted, but taking the weight divided by the geometric volume described by the shape. Unless noted, density refers to that of the overall structure and not the individual components, and will include in the measurement void volume of small pores and voids within the overall structure.

As used herein, the term “encapsulation” means the surrounding off or “caging” of a compound using a physical or chemical component.

As used herein, the term “inhibit” to prevent the normal growth of an organism or the activity of an enzyme or protein. As follows. “inhibitor” is any agent that prevents the normal growth of an organism or the activity of an enzyme or a protein.

The term “interlabial pad” refers to an absorbent product intended for the absorption of menstrual fluid or urine from the vaginal area by placement within the outer opening of the vagina. The interlabial pad comprises a liquid pervious topsheet, liquid impervious backsheet and an absorbent core disposed between the topsheet and the backsheet. Examples of such devices are described in U.S. Pat. No. 2,917,049 issued to Delaney on Dec. 15, 1959, U.S. Pat. No. 3,420,235 issued to Harmon on Jan. 7, 1969, U.S. Pat. No. 4,595,392 issued to Johnson, et al. on Jun. 17, 1986, and U.S. Pat. No. 5,484,429 issued to Vukos, et al. on Jan. 16, 1996. A commercially available interlabial device is the INSYNC Miniform interlabial pad which is marketed by A-Fem of Portland, Oreg. and described in U.S. Pat. Nos. 3,983,873 and 4,175,561 issued to Hirschman on Oct. 5, 1976 and Nov. 27, 1979, respectively.

The term “joined” or “attached,” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

The term “overwrap” refers to the external surface of a disposable article such as a sanitary napkin, pantiliner, interlabial device, tampon, disposble diapers, and the like. In tampon embodiments, the overwrap typically comprises a fluid permeable layer that surrounds the absorbent tampon's absorbent structure and is the portion, which is direct contact with the vaginal lining during use.

As used herein, the terms “pantiliner,” and “sanitary napkin,” refers to absorbent articles worn external about the pudenal region for the absorption of fluid therefrom, to aid in wound healing, or for the delivery of active materials, such as medicaments, or moisture. Sanitary napkins typically comprise a liquid pervious topsheet, liquid impervious backsheet and an absorbent core disposed between the topsheet and the backsheet. The sanitary napkin, as well as each layer or component thereof can be described as having a “body facing” surface and a “garment facing” surface. Pantiliners and sanitary napkin may have side extensions commonly referred to as “wings,” designed to wrap the sides of the crotch region of the panties of the user of sanitary napkin that may be extension of the topsheet and/or the backsheet. Such devices are disclosed in U.S. Pat. No. 4,463,045 issued to Ahr et al., 4,556,146 issued to Swanson et al., U.S. Pat. No. 4,950,264 issued to Osborn III, et al. and U.S. Pat. No. 4,687,478 issued to Van Tillburg.

By “pharmaceutically-acceptable carrier” as used herein is meant one or more compatible solid or liquid filler diluents, or encapsulating substances. By “compatible” as used herein is meant that the components of the composition are capable of being commingled without interacting in a manner which would substantially decrease the pharmaceutical efficacy of the total composition under ordinary use situations. Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations. Wetting agents and lubricants such as sodium lauryl sulfate, as well as colouring agents, flavouring agents, lubricants, excipients, tabletting agents, stabilizers, anti-oxidants such as ascorbic acid and vitamin E and preservatives, can also be present.

By “safe and effective amount” as used herein is meant a concentration high enough to significantly-positively modify the condition to be treated but low enough to avoid serious side effects (at a reasonable benefit/risk ratio), within the scope of sound medical judgment. A safe and effective amount of lactobacillus will vary with the particular condition being treated, the age and physical condition of the patient being treated, the severity of the condition, the duration of treatment, and the nature of concurrent therapy.

As used herein, a tampon has a “self-sustaining shape” when a tampon pledget has been compressed and/or shaped such that it assumes a general shape and size, which is vaginally insertable, absent external forces. It will be understood by one of skill in the art that this self-sustaining shape need not, and preferably does not persist during actual use of the tampon. That is, once the tampon is inserted and begins to acquire fluid, the tampon may begin to expand and may lose its self-sustaining form.

As used herein, the term “tampon,” refers to any type of absorbent structure that is inserted into the vaginal canal or other body cavities for the absorption of fluid therefrom, to aid in wound healing, or for the delivery of active materials, such as medicaments, or moisture. The tampon may be compressed into a generally cylindrical configuration in the radial direction, axially along the longitudinal axis or in both the radial and axial directions. While the tampon may be compressed into a substantially cylindrical configuration, other shapes are possible. These may include shapes having a cross section that may be described as rectangular, triangular, trapezoidal, semi-circular, hourglass, serpentine, or other suitable shapes. Tampons have an insertion end, withdrawal end, a length, a width, a longitudinal axis, a radial axis and an outer surface. The tampon's length can be measured from the insertion end to the withdrawal end along the longitudinal axis. A typical compressed tampon for human use is 30-60 mm in length. A tampon may be straight or non-linear in shape, such as curved along the longitudinal axis. A typical compressed tampon is 8-20 mm wide. The width of a tampon, unless otherwise stated in the specification, corresponds to the length across the largest cylindrical cross-section, along the length of the tampon.

The term “vaginal cavity,” “within the vagina,” and “vaginal interior,” as used herein, are intended to be synonymous and refer to the internal genitalia of the human female in the pudendal region of the body. The term “vaginal cavity” as used herein is intended to refer to the space located between the introitus of the vagina (sometimes referred to as the sphincter of the vagina or hymeneal ring,) and the cervix. The terms “vaginal cavity,” “within the vagina” and “vaginal interior,” do not include the interlabial space, the floor of vestibule or the externally visible genitalia.

The term “urogenital” as used herein, are intended to be synonymous and refer to the perineum, vulva, labia majora, all tissues enclosed by the labia majora including the labia minora, clitoris, introitus, fourchette, hymenal remnants, the vestibule and all major (e.g. Bartholin's) and minor vestibular glands, all sebaceous glands, the urethra and periurethral glands (e.g. Skene's glands) and internal organs including the urethra, ureters, and bladder.

The term “cfu” as used herein, are intended to refer to its common technical meaning as number of microbial colony forming units.

The term “gastrointestinal” as used herein, are intended to be synonymous and refer to the oral cavity, esophagus, stomach, small intestines, large intestines, colon, anus and perianal region.

The term “nasal” as used herein, are intended to be synonymous and refer to the nose, sinus and connecting cavities.

The term “wipes” as used herein refers to a substrate used for the absorption of fluid from the body, to aid in wound healing, or for the delivery of active materials, such as medicaments, or moisture.

The present invention relates to a composition and method for reducing human malodor. The composition and method comprise one or more species of bacteria.

The composition for reducing body odor in a human is selected from Lactobacillus iners, and all clones with at least 97% sequence similarity to Lactobacillus iners. The degree of similarity is determined by sequence similarity of the 16S rRNA genes. Methods for determining the sequences and the degree of similarity are described by Pavlova S I et. al. in J. Appl. Microbiol. 202;202;92(3)451-9 and by Zhou et. al. Microbiology. 203 Aug;150(pt 8):2565-73. The composition may further comprise Lactobacillus crispatus. The composition may also further comprise one or more species of bacteria selected from the group consisting of Lactobacillus casei, Lactobacillus gasseri, Lactobacillus fermentum, Lactobacillus amylolyticus, Lactobacillus acidophilus, Lactobacillus casei subs. pseudoplantarum, Lactobacillus brevis, Lactobacillus salivarius, Lactobacillus acidophilus, Lactobacillus plantarum, Lactobacillus fermentum, Lactobacillus jensenii, Lactobacillus coleohominis, Lactobacillus vaginas, Anaerococcus spp., Dialister spp., Finegoldia magna, Bifidobacterium spp., Bacteroides, thetaiotaomicron, Lachnospiraceae spp., Leptotrichia spp., Streptococcus spp., Hydrogenophaga palleronii, Comamonas spp., Aerococcus, spp., Veillonella, spp., Mycoplasma spp., and Micromonas spp.

The composition can comprise a safe and effective amount of one or more of the aforementioned bacteria with a pharmaceutically acceptable carrier.

This invention is not intended to be limited to any particular mode of application. Therefore oral, intravaginal, intraurethral or periurethral applications of the compositions can be used. The composition can be administered or applied in the form selected from the group consisting of a cream, paste, gum, a suppository, douche, mucoadehsive, liquid dental transport medium, moist wipe, microspheres, an ointments, an oral tablet, a liquid, a drink, a gel, and nasal spray.

One vehicle for delivery of beneficial bacteria may be microspheres comprised of poly (D.L-lactide-co-glycolide)(PLGA) and poly(D,L-lactide)(PLA) micropheres as described in Goodman, et al, Microsheres Under In Vitro Release Conditions, APPS PharmSCiTech, 2003: 4(4) article 50. Other methods for delivery or other mucoadhesives are described in U.S. Pat. No. 6,509,028 issued to Williams, et. al on Jan. 21, 2003. Another vehicle for delivery of a beneficial bacteria is an anaerobic dental transport medium available commercially from Anaerobe Systems, Morgan Hill, Calif.

Some forms of the composition may comprise one or more bacteria in a jelly base, preferably a K-Y jelly base. Another application involves the preparation of a freeze-dried capsule comprising the composition of the present invention. Effective dosages may range from 10³ to 10¹³ cfu per daily dose and more preferably from 10⁵ to 10¹⁰ cfu/ml per daily dose. Typically effective dosages are in the range of 10⁹ cfu/ml.

The treatment method may vary according to the individual condition of the subject. For example, one regimen involves the subject taking a continuous self administered dose one or more times a day. Another regimen involves the subject self administering a single dose at least once per week on an on-going basis. Yet another regiment involves the subject self administering one or more doses for a period of 1 to 120 days.

The method for reducing human malodor comprises administering one or more bacteria selected from the group consisting of Lactobacillus iners and all clones with at least 97% sequence similarity to Lactobacillus iners. The degree of similarity is determined by sequence similarity of the 16S rRNA genes.

The composition may further comprise Lactobacillus crispatus. The composition may comprise one or one species of bacteria selected from the group consisting of Lactobacillus casei, Lactobacillus gasseri, Lactobacillus fermentum, Lactobacillus amylolyticus, Lactobacillus acidophilus, Lactobacillus casei subs.pseudoplantarum, Lactobacillus brevis, Lactobacillus salivarius, Lactobacillus acidophilus, Lactobacillus. plantarum, Lactobacillus fermentum, Lactobacillus jensenii, Lactobacillus coleohominis, Lactobacillus vaginas, Anaerococcus spp., Dialister spp., Finegoldia magna, Bifidobacterium spp., Bacteroides thetaiotaomicron, Lachnospiraceae spp, Leptotrichia spp,., Streptococcus spp., Hydrogenophaga palleronii, Comamonas spp., Aerococcus, spp., Veillonella, spp, Mycoplasma spp., and Micromonas spp.

The method may comprise applying the composition directly to the urogenital region of a human with a device selecting from the group consisting of tampons, pantiliners, sanitary pad, interlabial pad, overwrap, wipes, and pessaries.

Although the present invention is not bound by any one theory or mode of operation, it is believed that, at least to some degree, that the inclusion of lactobacillus iners with other lactobacillus species provide the opportunity for the body to re-establish a healthy flora by reducing or excluding the population of pathogenic bacteria in the vagina, urinary tract, gastrointestinal tract and nasal area. By re-establishing a healthy flora malodor associated with an unhealthy flora can be reduced and even eliminated. From the standpoint of physical exclusion, the attachment of Lactobacillus acts as a block to uropathogens by preventing access to receptor sites. Although complete exclusion of uropathogens theoretically can occur, the most common finding of the results of the present invention is that there is a reduction in uropathogen numbers compared to lactobacilli. In other words, although some lactobacilli may not completely exclude uropathogens, they are still capable of interfering with uropathogen colonization in vivo. Coaggregation is an important element as it allows lactobacilli to form a urogenital mixed flora present in healthy patients. This mixed flora is preferably dominated by lactobacilli and other indigenous gram positive bacteria. It is hypothesized that the lactobacilli of the present invention and some uropathogens coaggregate (Reid et al. 1988, Can. J. Microbiol. 34:344-351, the entire contents of which are incorporated herein by reference), in a way that interferes with the pathogenic process.

The compositions of the present invention may include a growth factor for facilitating the growth of lactic acid bacteria. The phrase “a growth factor for facilitating the growth of lactic acid bacteria,” as used herein is meant a nutrient source or media which supplies a necessary source of food and/or energy for facilitating the growth of lactic acid producing bacteria. The growth factor is preferably selective for establishing and maintaining the growth of lactic acid bacteria, preferably Lactobacillus and/or Bifidobacterium, without facilitating extreme growth of pathogenic bacteria. The various nutritional requirements essential for bacterial and/or colony growth are normally met when the growth factor contain fermentable carbohydrate, peptone, meat and yeast extract. Supplementations with tomato juice, manganese, acetate and oleic acid esters, especially Tween 80, are stimulatory or even essential for most species and are, therefore, included in most MRS medium. Lactic acid bacteria adapted to very particular substrates may require special growth factors.

Examples of suitable growth factors include, but are not limited to, yeast extracts; gangliosides; salicin; mono-, di- and polysaccharide sugars such as glycogen, glucose, fructose, rharnnose, lactulose, methyl-a-D-mannoside, p-nitrophenol-cc-D-mannoside, maltose, maltodextrin, dextrin, dextran, levan, sialic acid and acetylglucosamine as well as oligosaccharides such as, but not limited to, fructooligosaccharides, galactooligosaccharides and soybean oligosaccharides. Fiber or fermentable substrates such as psyllium may be used in the present compositions as may gums such as guar gum and xanthum gum. Similarly, proteinacious materials such as, peptone, keratin; vegetable; soy and unsaturated fatty acids such as lauric acid and teichoic acids such as lipoteichoic acid and esters such as glycerophosphates or P-glycerophosphates are also useful as growth factors. The growth factor is preferably selected for establishing and maintaining the growth of lactic acid bacteria, most preferably Lactobacillus and/or Bifidobacterium species. Growth factors preferable for use in the compositions of the present invention include lactose, lactulose, rhanmose, oligosaccharides and glycogen. Mixtures of these 15 nutrients may also be used.

More preferably the growth factor of the present invention is an oligosaccharide such as, but not limited to, galactooligosaccharides, soybean oligosaccharides and fructooligosaccharides. Oligosaccharides possess bioadhesive properties which help fix the location of these growth factors for easier access by lactic acid bacteria. Most preferred for use herein are fructooligosaccharides. Lactic acid bacteria, such as Lactobacillus and Bifidobacterium, partially utilize fructooligosaccharides as an energy source by converting it, via fermentation, to lactic acid or a mixture of lactic acid, acetic acid, and CO₂. The lactic acid and other fatty acids produced by this carbohydrate fermentation contribute to the maintenance of low pH which is an important control mechanism for preventing colonization of pathogens.

Chemically, oligofi-uctose is the oligosaccharide fraction of inulin. It is composed of the GFn and Fn type [G=glucose; F=fructose; n=number of fi-utose moieties linked by 0 (2, 1) linkages in a ratio of about 2: 1, with n=2−6, and an average degree of polymerization of 4. Inulin is prepared by hot water extraction of chicory roots and is composed of molecules of the GFn type, n ranging as high as 60 with an average degree of polymerization of 10. Fructooligosaccharides suitable for use herein may or may not have non-fiructosyl units in place of fructosyl end units. The same is true for other oligosaccharides with respect to their osyl end units. Non-fructosyl units may include, but are not limited to, polyalcohols such as xylitol, mannitol, and sorbitol.

Fructooligosacchafides most preferred for use in the present Compositions are inulin or oligofructose. Mixtures of these nutrients may also be used.

The present invention may also be useful in maintaining and restoring normal flora of the gastrointestinal tract, nasal passages and urogenital region of men and women and help treat or prevent human malodor from the mucosal surfaces of the body. Such mucosal surfaces include the mouth, nose, vagina, urogenital region, gastrointestinal tract.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition for reducing human malodor comprising one or more bacteria selected from the group consisting of Lactobacillus iners and all clones with at least 97% sequence similarity to Lactobacillus iners as determined by sequences from 16S rRNA genes.

2. The composition of claim 1, wherein said composition further comprises Lactobacillus crispatus.

3. The composition of claim 1, wherein said composition further comprises one or more bacteria selected from the group consisting of Lactobacillus casei, Lactobacillus gasseri, Lactobacillus fermentum, Lactobacillus amylolyticus, Lactobacillus acidophilus, Lactobacillus casei subs. pseudoplantarum, Lactobacillus brevis, Lactobacillus salivarius, Lactobacillus acidophilus, Lactobacillus plantarum, Lactobacillus fermentum, Lactobacillus jensenii, Lactobacillus crispatus, Lactobacillus vaginalis, Lactobacillus mucosae, Lactobacillus paracasei, Lactobacillus rhamnosus, Lactobacillus coleohominis Lactobacillus vaginas, Anaerococcus spp., Bifidobacterium spp., Bacteroides thetaiotaomicron, Dialister spp., Finegoldia magna, Lachnospiraceae spp., Leptotrichia spp., Streptococcus spp., Hydrogenophaga palleronii, Comamonas spp., Aerococcus, spp., Veillonella, spp., Mycoplasma spp., and Micromonas spp.

4. The composition according to claim 1 may be administered as a suppository, douche, mouth wash, oral tablet, capsule, drink, gum, nasal spray, pad, liner, interlabial device, wipe, pessary, tampon or nasal packing.

5. The composition according to claim 1 wherein said composition is in the form selected from the group consisting of a cream, paste, gum, a suppository, mucoadhesive, liquid dental transport medium, microspheres, an ointment, an oral tablet, a liquid, and a gel.

6. The compositions of claim 1 further comprises a growth factor.

7. A method for reducing human malodor comprising administering a safe and effective amount of one or more bacteria selected from the group consisting of Lactobacillus iners, and all clones with at least 97% sequence similarity to Lactobacillus iners as determined by sequences from 16S rRNA genes.

8. The method of claim 7 wherein each bacteria is administered in a dose of from about 10³ to about 10¹³ cfu/ml.

9. The method of claim 7 wherein each bacteria is administered in a dose of from about 10⁵ to about 10¹⁰ cfu/ml.

10. The method according to claim 7, wherein said composition further comprises Lactobacillus crispatus.

11. The method according to claim 7, wherein said composition further comprises one or more species of bacteria selected from the group consisting of Lactobacillus casei, Lactobacillus gasseri, Lactobacillus fermentum, Lactobacillus amylolyticus, Lactobacillus acidophilus, Lactobacillus casei subs. pseudoplantarum, Lactobacillus brevis, Lactobacillus salivarius, Lactobacillus acidophilus, Lactobacillus. plantarum, Lactobacillus fermentum, Lactobacillus jensenii. Lactobacillus crispatus, Lactobacillus vaginalis, Lactobacillus mucosae, Lactobacillus paracasei, Lactobacillus rhamnosus, Lactobacillus coleohominis, Lactobacillus vaginas, Anaerococcus spp., Dialister spp., Finegoldia magna, Bacteroides thetaiotaomicron, Bifidobacterium spp., Lachnospiraceae spp., Leptotrichia spp., Streptococcus spp., Hydrogenophaga palleronii, Comamonas spp., Aerococcus, spp., Veillonella, spp, Mycoplasma spp., and Micromonas spp.
